(19) United States
(12) Patent Application Publication
(10) Pub. No.: US 2023/0233812 A1
(43) Pub. Date: Jul. 27, 2023

(54) IV CATHETER DEVICE HAVING A NEEDLE COVER THAT FUNCTIONS AS A NEEDLE SHIELD

(71) Applicant: Becton, Dickinson and Company, Franklin Lakes, NJ (US)

(72) Inventors: Deepan Vikash, Tamil Nadu (IN); Archit Thiagarajan, Coimbatore (IN); Sakthivel Karthikeyan, Tamil Nadu (IN)

(21) Appl. No.: 18/094,776

(22) Filed: Jan. 9, 2023

Related U.S. Application Data
(60) Provisional application No. 63/302,902, filed on Jan. 25, 2022.

(51) Int. Cl. A61M 25/06

(52) U.S. Cl. CPC A61M 25/0631; A61M 25/065; A61M 25/0606

(57) ABSTRACT

An IV catheter device can include needle cover that functions as a needle shield. By using the needle cover as a needle shield, the design of the IV catheter device is simplified thereby reducing its cost. The needle cover can be configured to slide along a needle hub between a distalmost position in which the needle cover functions as either a needle cover or a needle shield and a proximal position in which the needle cover exposes the needle to enable the catheter to be inserted into a patient's vasculature.

### RELATED APPLICATIONS

This application claims the benefit of U.S. Provisional Patent Application No. 63/302,902, filed on Jan. 25, 2022, and entitled IV CATHETER DEVICE HAVING A NEEDLE COVER THAT FUNCTIONS AS A NEEDLE SHIELD, which is incorporated herein in its entirety.

### BACKGROUND

Intravenous (IV) catheter devices are commonly used for a variety of infusion therapies. For example, an IV catheter device may be used for infusing fluids, such as normal saline solution, various medicaments, and total parenteral nutrition, into a patient. IV catheter devices may also be used for withdrawing blood from the patient.

A common type of IV catheter device is an over-the-needle peripheral intravenous catheter (“PIVC”). As its name implies, the over-the-needle catheter may be mounted over a needle having a sharp distal tip. The catheter and the needle may be assembled so that the distal tip of the needle extends beyond the distal tip of the catheter with the bevel of the needle facing up away from skin of the patient.

IV catheter devices oftentimes include needle shields that secure the sharp distal tip of the needle after it is withdrawn from the catheter to prevent accidental needle sticks. These needle shields are typically made of metal and are complex thereby increasing the cost of the IV catheter device. IV catheter devices also oftentimes include a needle cover, which is separate from the needle shield, that covers the sharp distal tip of the needle before it is used to insert the catheter into a patient's vasculature. In other words, the needle cover is typically a simple component, like a cap, that is removed and discarded before inserting a catheter, whereas the needle shield is typically a complex component that secures the distal tip of the needle after it is withdrawn from the patient's vasculature and the catheter adapter.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to IV catheter devices that have a needle cover that also functions as a needle shield. By using the needle cover as a needle shield, the design of the IV catheter device is simplified thereby reducing its cost. The needle cover can be configured to slide along a needle hub between a distalmost position in which the needle cover functions as either a needle cover or a needle shield and a proximal position in which the needle cover exposes the needle to enable the catheter to be inserted into a patient's vasculature.

In some embodiments of the present disclosure, an IV catheter device may include a catheter assembly, a needle assembly and a needle cover. The catheter assembly may have a catheter adapter and a catheter that extends distally from the catheter adapter. The needle assembly may have a needle hub and a needle that extends distally from the needle hub. The needle hub may be coupled to the catheter adapter such that the needle extends through the catheter. The needle cover may be coupled to the needle hub and may configured to function as a needle shield after the needle is withdrawn from the catheter adapter.

In some embodiments, the needle cover may be coupled to the needle hub via one or more channels and corresponding one or more guides.

In some embodiments, the one or more channels may extend along the needle hub and the corresponding one or more guides may be formed on the needle cover.

In some embodiments, each of the one or more channels may include a distal stop and a retaining bump spaced from the distal stop.

In some embodiments, the retaining bump may prevent the corresponding guide from sliding proximally within the channel.

In some embodiments, each of the one or more channels may include a proximal stop.

In some embodiments, each of the one or more channels may include a locking slot into which the one or more guides are rotated.

In some embodiments, the needle hub may include one or more entries corresponding to the one or more channels by which the one or more guides enter the one or more channels.

In some embodiments, the needle cover may include a channel and the catheter adapter may include a push tab that extends through and slides within the channel.

In some embodiments, the channel may include a deviation.

In some embodiments, the needle hub may include one or more cutouts and the needle shield may include one or more support tabs that insert through the one or more cutouts to assemble the needle hub and needle shield.

In some embodiments, the channel in the needle cover may extend from a proximal end to a distal end of the needle cover.

In some embodiments, the needle cover may include one or more grips.

In some embodiments of the present disclosure, an IV catheter device may include a catheter assembly, a needle assembly and a needle cover. The catheter assembly may have a catheter adapter and a catheter that extends distally from the catheter adapter. The needle assembly may have a needle hub and a needle that extends distally from the needle hub. The needle hub may be coupled to the catheter adapter such that the needle extends through the catheter. The needle cover may be coupled to the needle hub and may be configured to slide along the needle hub from a distalmost position to a proximal position. The needle cover may enclose the needle and the catheter when the needle cover is in the distalmost position and the catheter adapter is coupled to the needle hub. The needle cover may also enclose the needle when the needle cover is in the distalmost position and the catheter assembly has been slid distally out from the needle cover.

In some embodiments, the needle cover may include a channel and the catheter adapter may include a push tab that extends through the channel. The push tab may enable a clinician to slide the catheter adapter distally out from the needle cover.

In some embodiments, the needle hub may include one or more channels and the needle cover may include corresponding one or more guides that slide within the one or more channels.

In some embodiments, each of the one or more channels may include a retaining bump that interfaces with the corresponding guide to retain the needle cover in the distalmost position.

In some embodiments, each of the one or more channels may include a distal stop that is spaced from the retaining bump.

In some embodiments, when the needle cover is in the proximal position, the needle and the catheter may extend distally out from the needle cover.

In some embodiments, the catheter adapter may include a notch and the needle hub may include a protrusion that inserts into the notch to thereby limit rotation of the catheter adapter relative to the needle hub.

In some embodiments, the needle cover may include one or more grips that extend outwardly from a proximal end of the needle cover.

In some embodiments of the present disclosure, an IV catheter device may include a catheter assembly, a needle assembly and a needle shield. The catheter assembly may include a catheter adapter and a catheter that extends distally from the catheter adapter. The catheter adapter may include a push tab. The needle assembly may include a needle hub and a needle that extends distally from the needle hub. The needle hub may be coupled to the catheter adapter such that the needle extends through the catheter. The needle hub may have one or more channels that extend lengthwise along the needle hub. The needle cover may have one or more guides that are positioning within the one or more channels to thereby couple the needle cover to the needle hub. The one or more guides may be configured to slide within the respective channel. The needle cover may include a channel within which the push tab of the catheter adapter slides.

In some embodiments, each of the one or more channels may include a distal stop and a retaining bump. The respective guide may be selectively retained between the distal stop and the retaining bump.

In some embodiments, the needle cover encloses the needle after the needle has been withdrawn from the catheter adapter.

In some embodiments, each of the one or more channels may include a locking slot into which the respective guide is rotated.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

BRIEF DESCRIPTION OF THE SEVERAL VIEWS OF THE DRAWINGS

Example embodiments will be described and explained with additional specificity and detail through the use of the accompanying drawings in which:

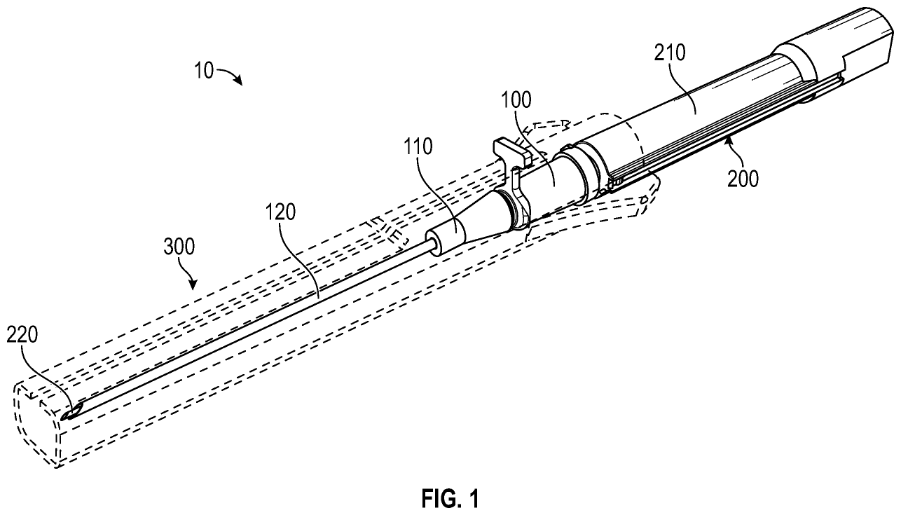

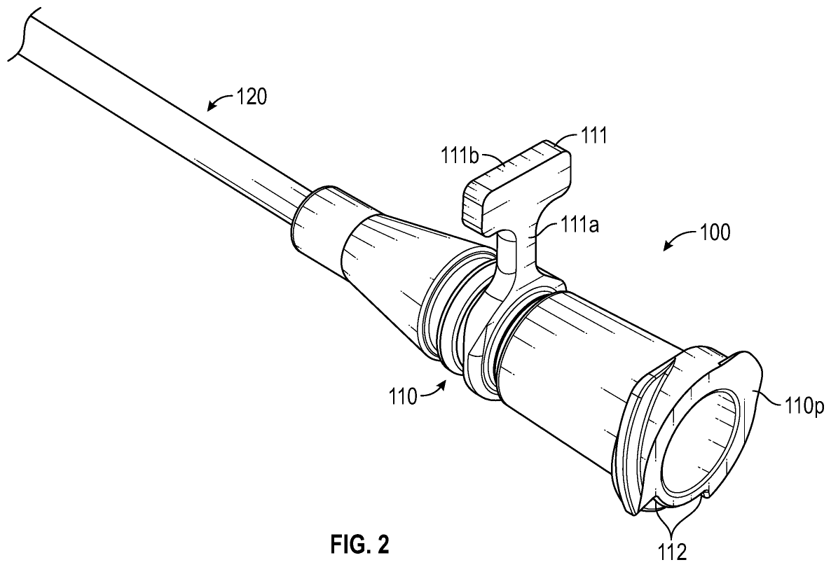

FIG. 1 is an example of an IV catheter device that is configured in accordance with one or more embodiments of the present disclosure;

FIG. 2 illustrates a catheter assembly of the IV catheter device of FIG. 1;

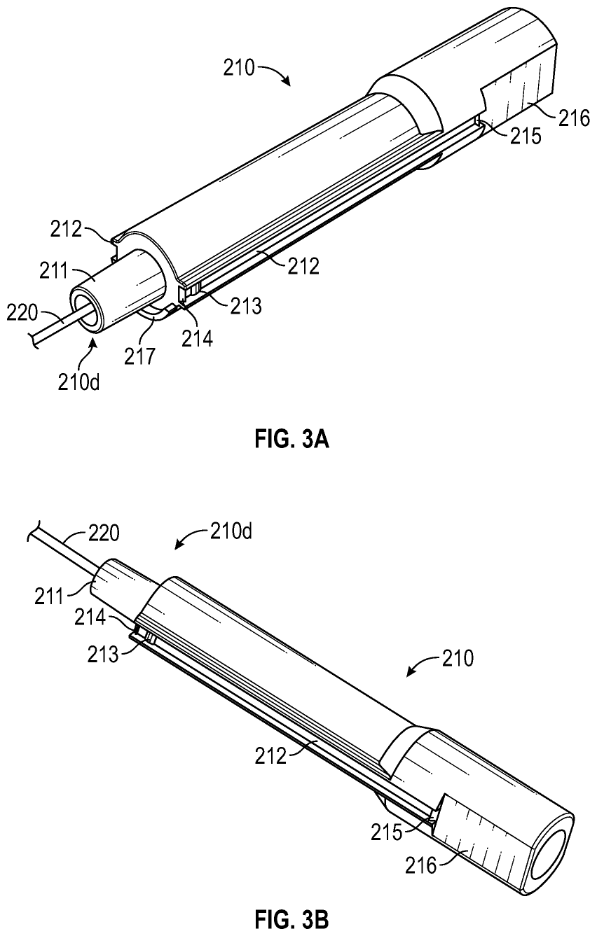

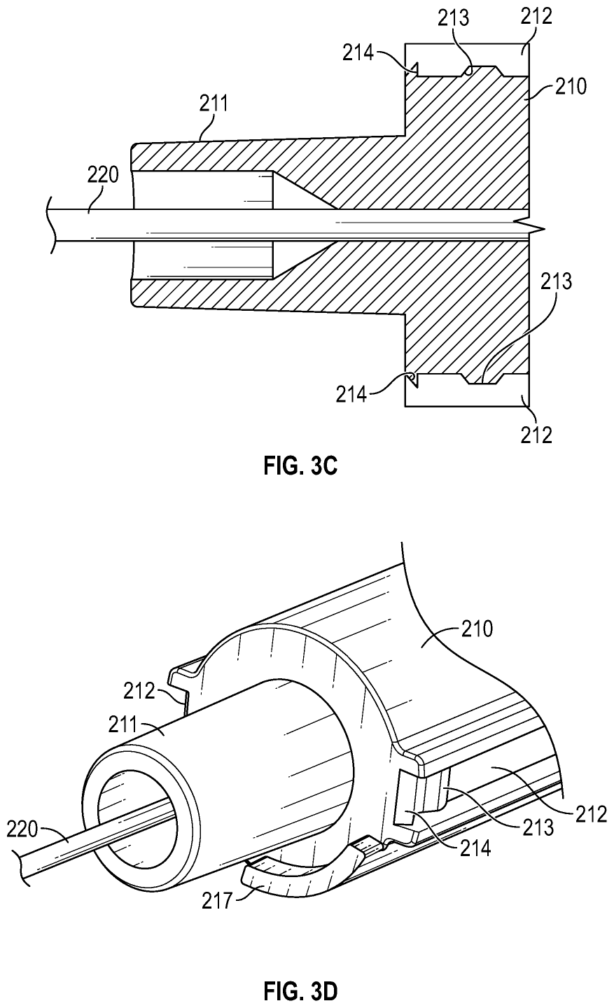

FIGS. 3A-3D are various views of a needle assembly of the IV catheter device of FIG. 1;

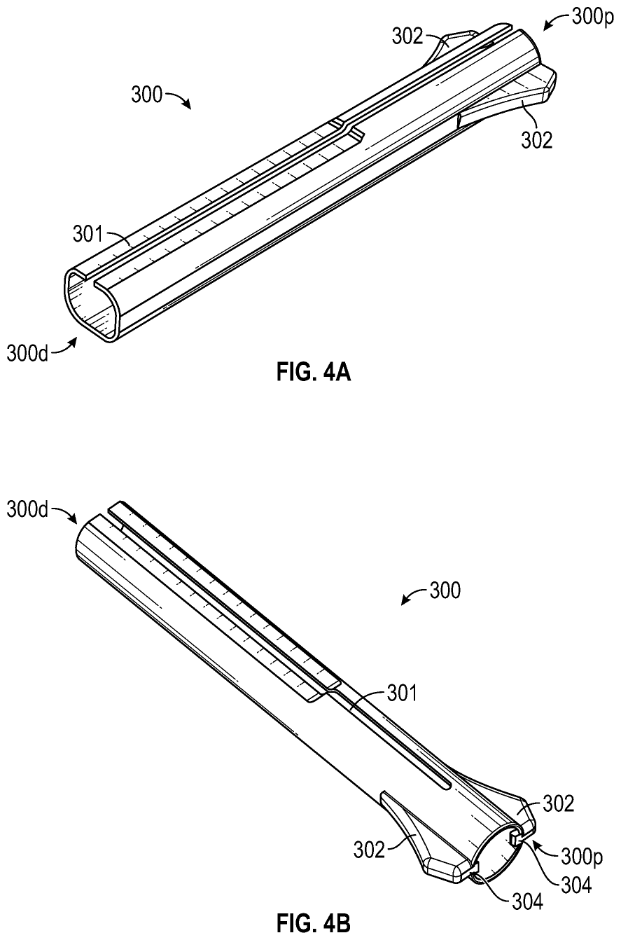

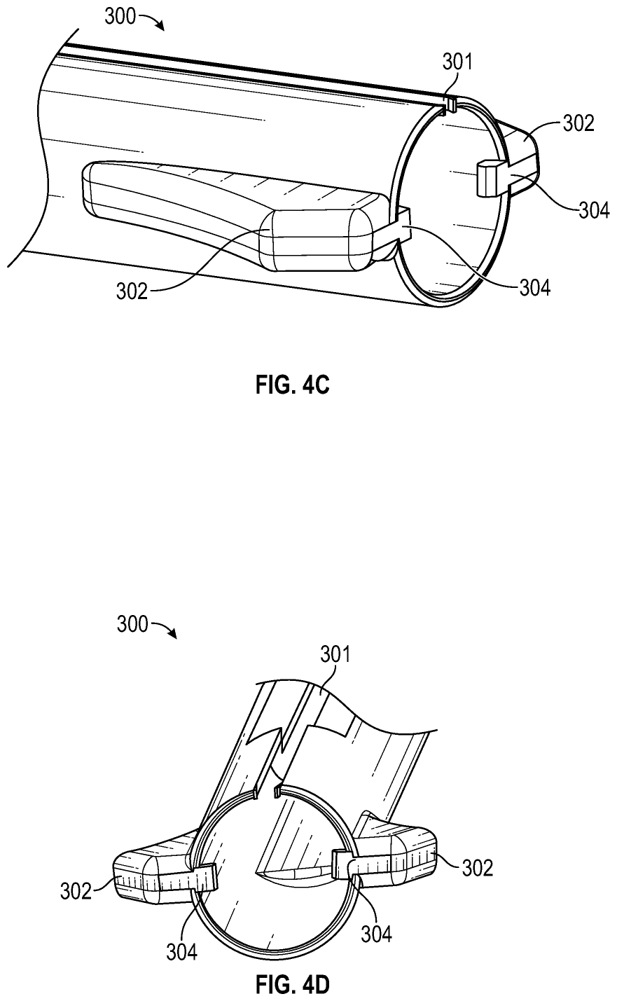

FIGS. 4A-4D are various views of a needle shield of the IV catheter device of FIG. 1;

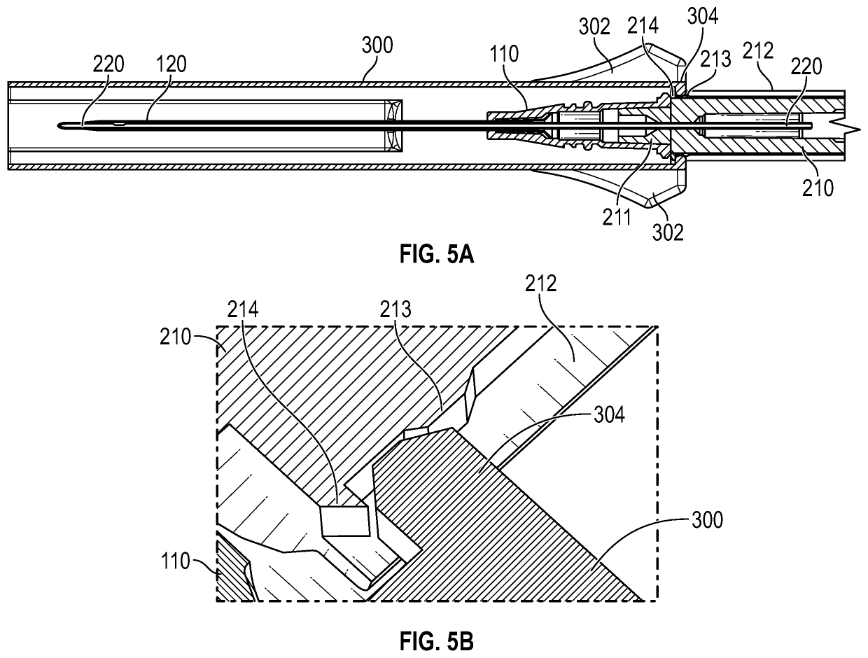

FIG. 5A is a cross-sectional view of the IV catheter device of FIG. 1;

FIG. 5B is a cross-sectional view of the needle assembly and the needle shield of the IV catheter device of FIG. 1;

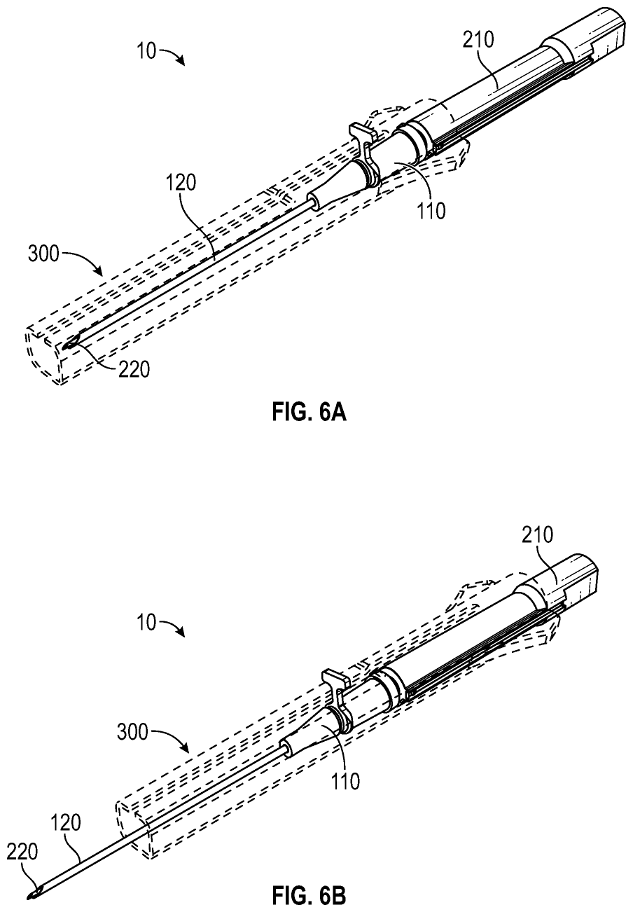

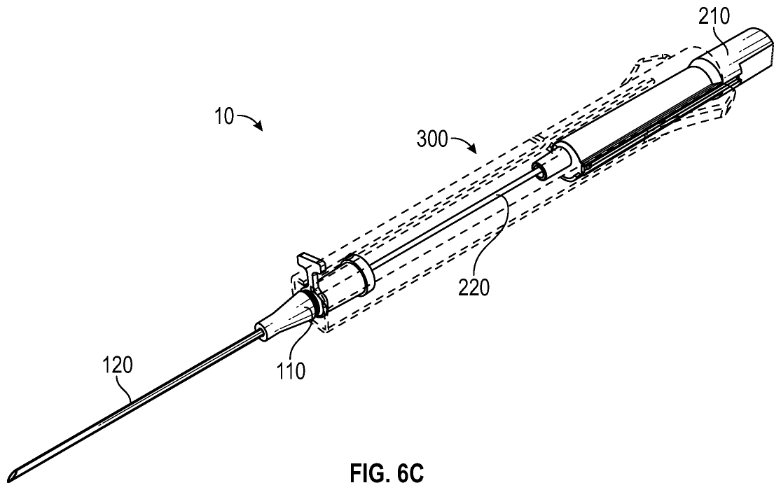

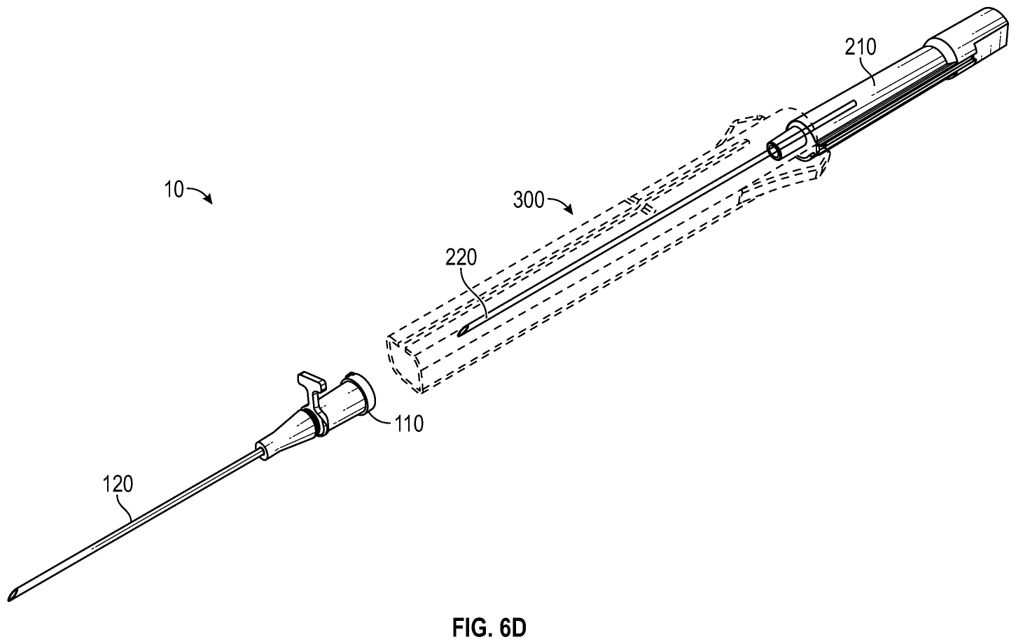

FIGS. 6A-6D provide an example of how the IV catheter device of FIG. 1 may be used;

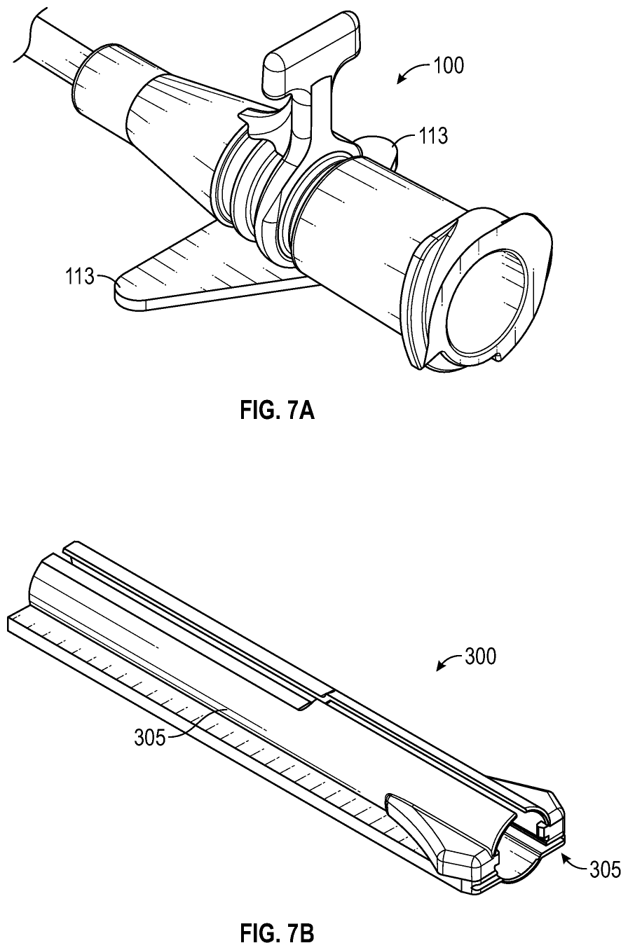

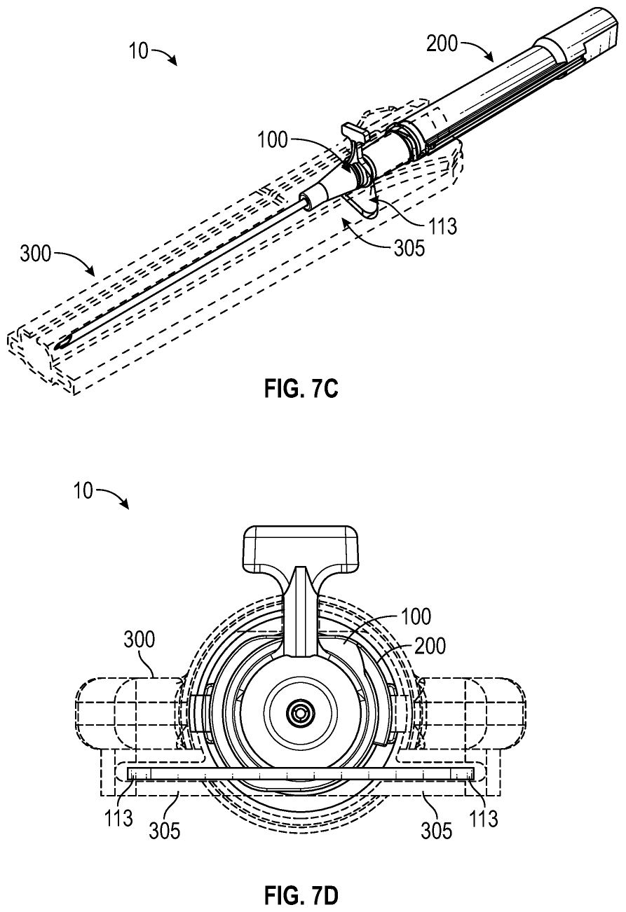

FIGS. 7A-7D provide another example of an IV catheter device that is configured in accordance with one or more embodiments of the present disclosure;

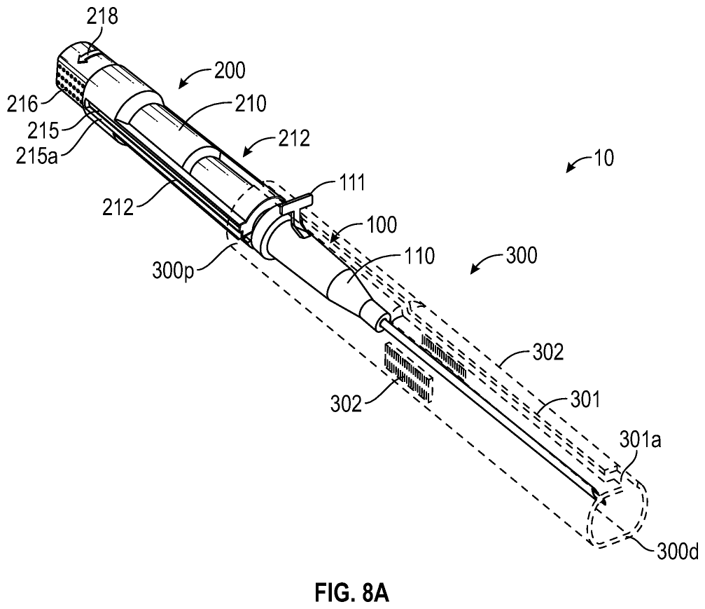

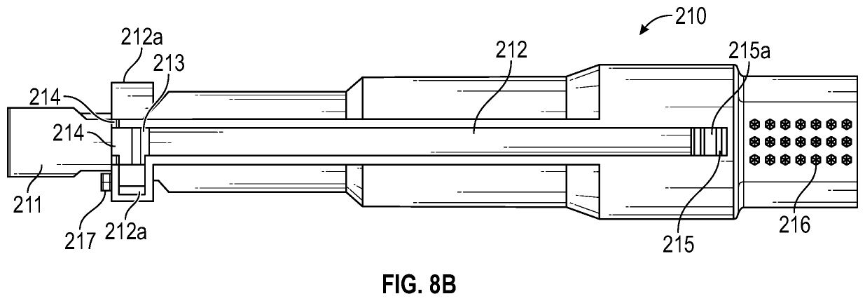

FIGS. 8A-8D provide another example of an IV catheter device that is configured in accordance with one or more embodiments of the present disclosure;

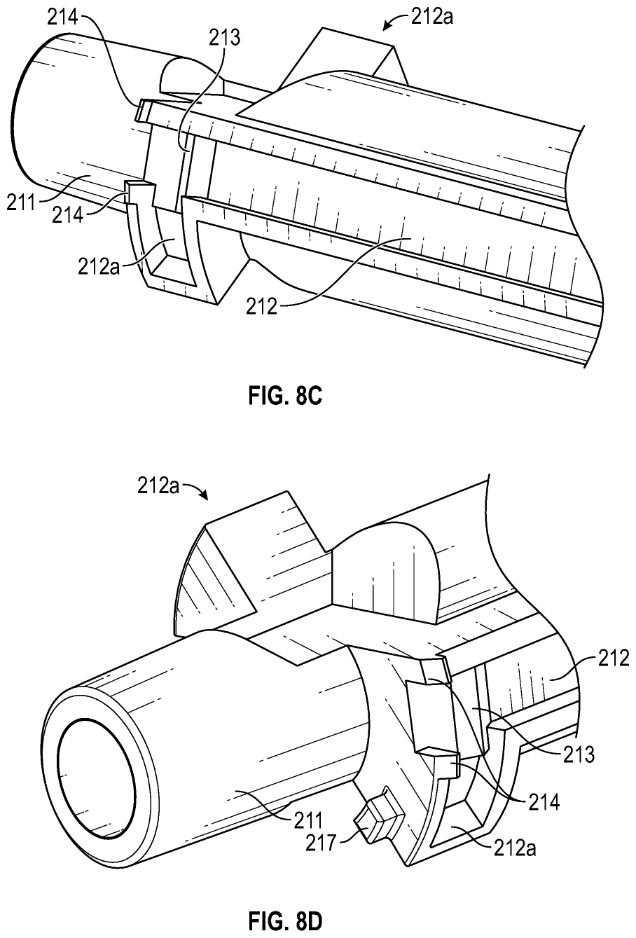

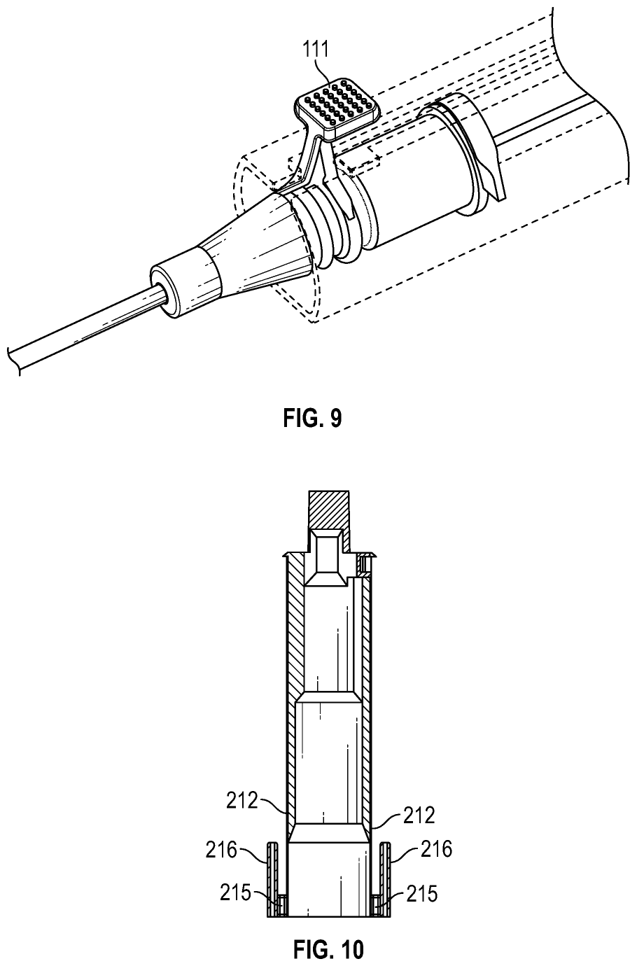

FIG. 9 provides an example of a push tab that may be used with an IV catheter device that is configured in accordance with one or more embodiments of the present disclosure;

FIG. 10 provides an example of gripping regions that may be used with an IV catheter device that is configured in accordance with one or more embodiments of the present disclosure;

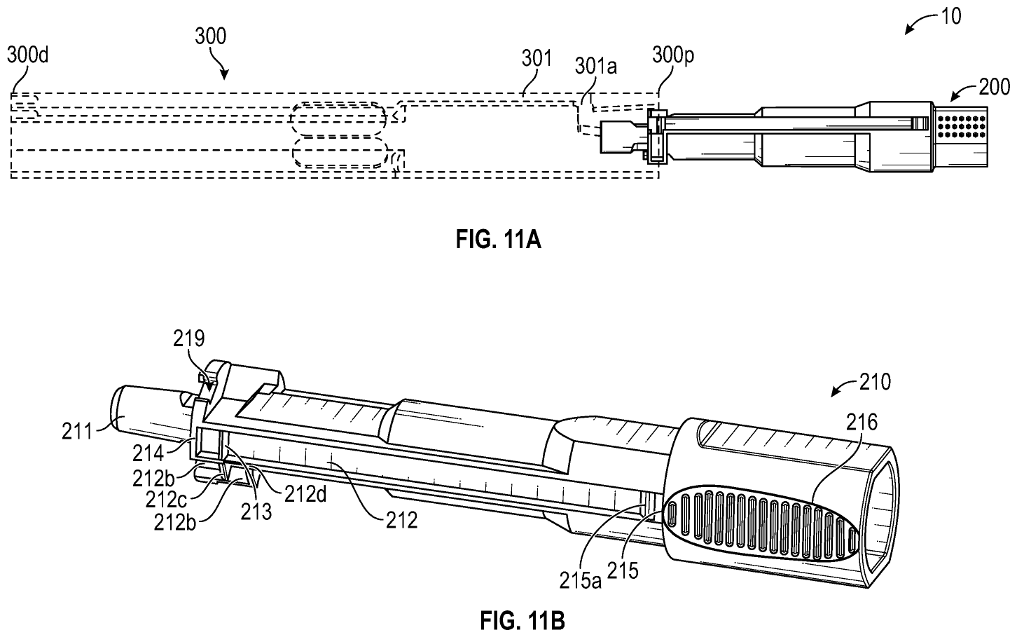

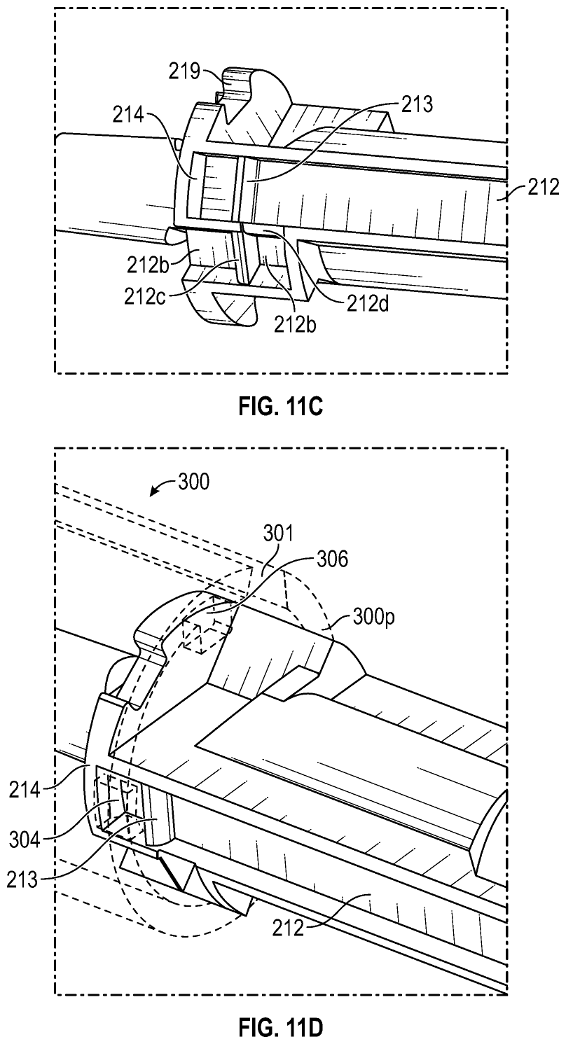

FIGS. 11A-11D provide another example of an IV catheter device that is configured in accordance with one or more embodiments of the present disclosure;

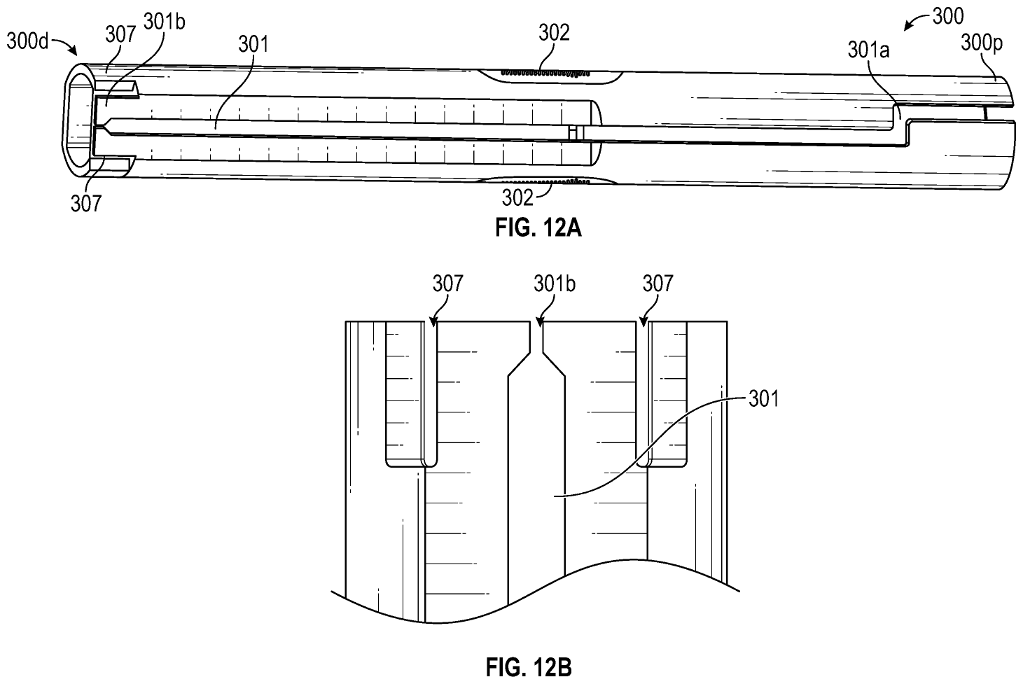

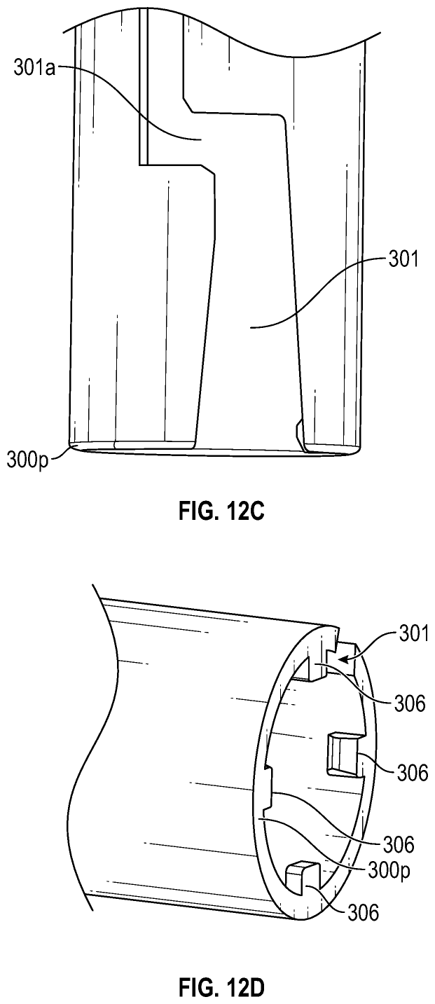

FIGS. 12A-12D provide an example of a needle shield that may be used with an IV catheter device that is configured in accordance with one or more embodiments of the present disclosure;

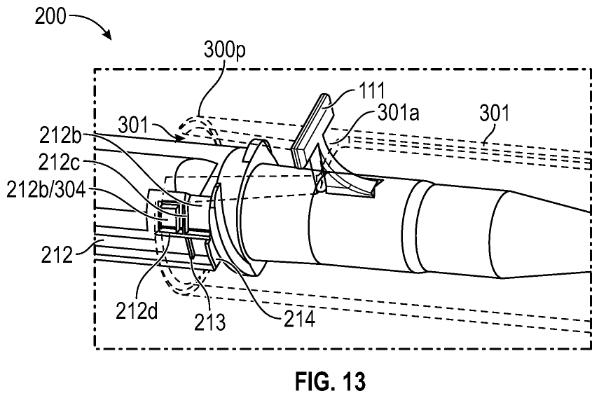

FIG. 13 provides an example of how an IV catheter device that is configured in accordance with one or more embodiments of the present disclosure may be assembled; and

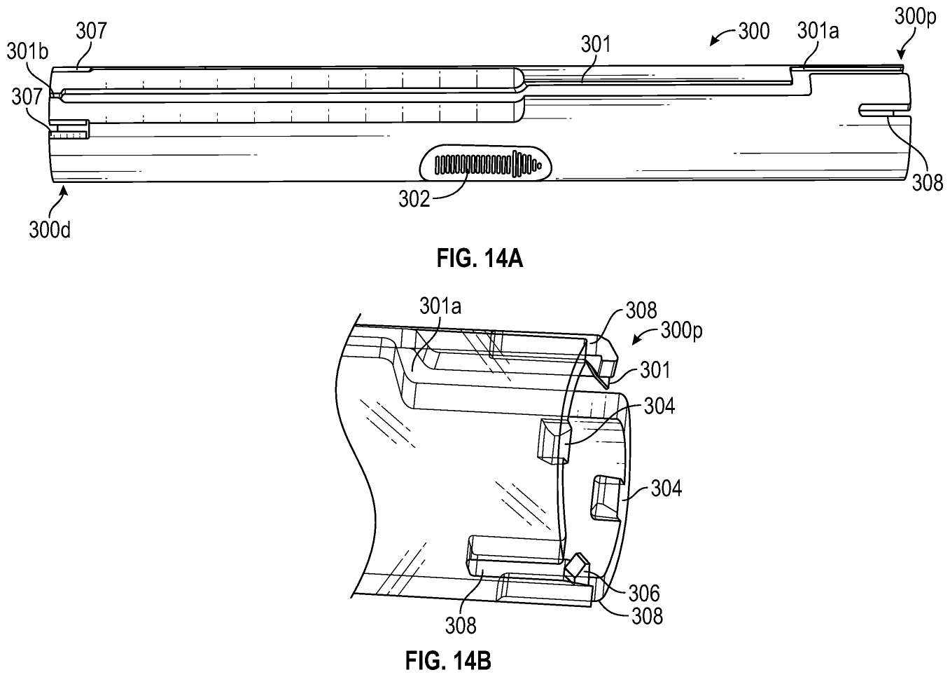

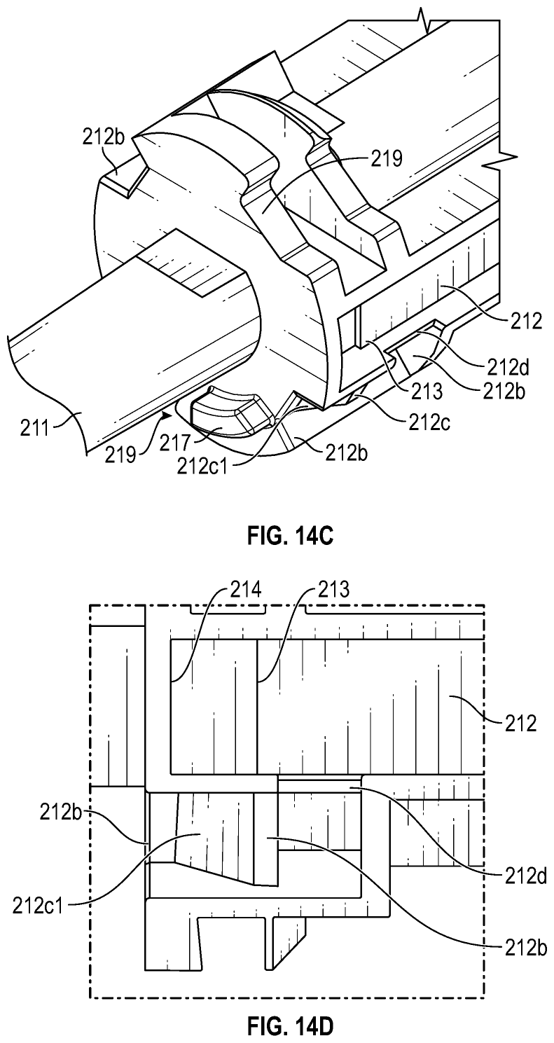

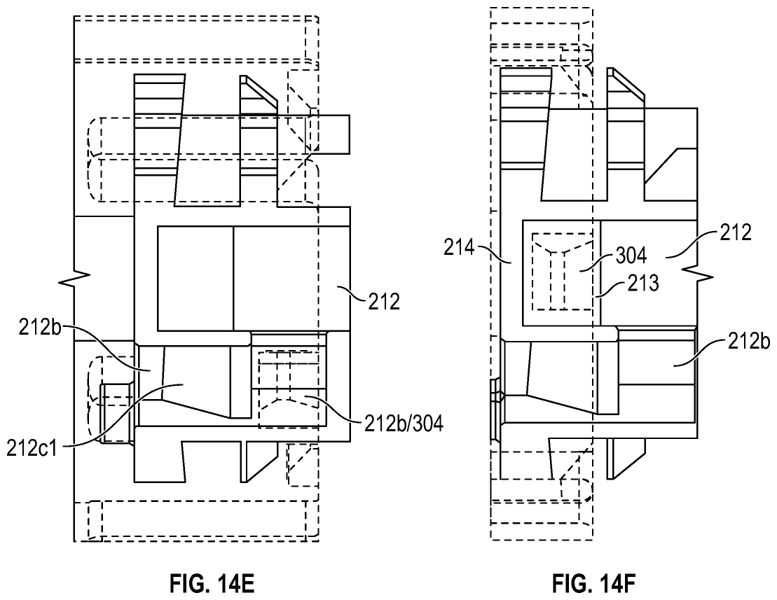

FIGS. 14A-14F provide another example of an IV catheter device that is configured in accordance with one or more embodiments of the present disclosure.

### DESCRIPTION OF EMBODIMENTS

An IV catheter device can include needle cover that functions as a needle shield. By using the needle cover as a needle shield, the design of the IV catheter device is simplified thereby reducing its cost. The needle cover can be configured to slide along a needle hub between a distalmost position in which the needle cover functions as either a needle cover or a needle shield and a proximal position in which the needle cover exposes the needle to enable the catheter to be inserted into a patient's vasculature.

FIG. 1 illustrates an IV catheter device 10 that is configured in accordance with one or more embodiments of the present disclosure. In FIG. 1, IV catheter device 10 is in a pre-use configuration. IV catheter device 10 includes a catheter assembly 100, a needle assembly 200 and a needle cover 300. Catheter assembly 100 includes a catheter adapter 110 and a catheter 120 that extends distally from catheter adapter 110. Needle assembly 200 includes a needle hub 210 and a needle 220 that extends distally from needle hub 210. In the pre-use configuration, needle hub 210 can be coupled to catheter adapter 110 such that needle 220 extends through and distally beyond catheter 120.

Needle shield 300, which can have an elongated, tubular shape, is coupled to needle hub 210 and extends overtop catheter adapter 110, catheter 120 and needle 220. Notably, in the pre-use configuration, the distal tip of needle 220 will be contained within needle cover 300.

As an overview, needle cover 300 can be configured to slide along needle hub 210 to allow needle cover 300 to function as a needle cover prior to insertion of catheter 120, and then as a needle shield when needle 220 is withdrawn from catheter adapter 110. Needle hub 210 and needle cover 300 can be configured to interlock to maintain the distal tip of needle 220 within needle cover 300 when it is functioning as a needle cover and as a needle shield. Needle cover 300 can also be configured to enable catheter adapter 110 to be slid within needle cover 300 when placing catheter 120 intravenously. Needle cover 300 provides integrated safety in that it secures the distal tip of needle 220 in response to needle 220 being withdrawn from catheter adapter 110. Also, needle cover 300 provides healthcare worker safety in that it covers the entirety of needle 220 when it functions as a needle shield. Needle cover 300 can provide these benefits with a simple design as described below.

FIG. 2 illustrates catheter assembly 100 in isolation. As shown, catheter adapter 110 can include a push tab 111 that extends upwardly from catheter adapter 110 and a notch 112 that is formed in proximal end 110p of catheter adapter 110. Push tab 111 can include a neck portion 111a and a tab portion 111b that has an expanded width relative to neck portion 111a. Push tab 111 can enable a clinician to advance catheter adapter 110 while catheter adapter 110 is enclosed within needle cover 300.

In some embodiments, a push tab of catheter adapter 110 could be in the form of opposing foldable wings as opposed to being fixed and rigid as in the depicted embodiments. For example, the opposing foldable wings of a push tab could be folded together in an upright direction to allow them to extend through channel 301 of needle cover 300 (as described below) and function as a push tab. Then, when catheter adapter 110 is removed from needle cover 300, the opposing foldable wings of the push tab can be folded into a flat orientation in which they extend outwardly from opposing sides of catheter adapter 110. In this flat orientation, the opposing foldable wings of the push tab can function as a securement platform for securing catheter adapter 110 to the patient's skin.

In other embodiments, push tab 111 could include extensions that may extend downwardly and outwardly from tab portion 111b so as to not interfere with needle cover 300 while also forming a securement platform. In some embodiments, catheter adapter 110 may include wings that are separate from push tab 111 and needle cover 300 may be adapted to accommodate the wings. FIGS. 7A-7D, which are described in detail below, provide an example of such embodiments.

FIGS. 3A-3D provide various views of needle assembly 200 in isolation. Needle hub 210 can include a distal end 210d that is configured to couple to proximal end 110p of catheter adapter 110. For example, distal end 210d may form a tapered tip 211 that inserts into proximal end 110p of catheter adapter 110 to form a friction fit. A protrusion 217 may extend distally from needle hub 210 alongside tapered tip 211 and may be shaped and sized to insert into notch 112 in proximal end 110p of catheter adapter 110 when tapered tip 211 is fully inserted into proximal end 110p. This engagement of protrusion 217 with notch 112 can prevent needle hub 210 from rotating relative to catheter adapter 110.

Needle hub 210 can also include one or more channels 212 that extend lengthwise along its outer surface. In the depicted embodiment, needle hub 210 includes two channels 212 that are spaced 180 degrees apart on opposite sides of needle hub 210. As described in detail below, needle cover 300 can be configured to slide along channels 212. A stop 214 can be formed in channel 212 at or towards its distal end and a stop 215 can be formed in channel 212 at or towards its proximal end. Stops 214 and 215 can prevent needle cover 300 from detaching from needle hub 210. In some embodiments, a distal side of stop 214 can be ramped to facilitate assembly. In particular, the ramped distal side can facilitate sliding needle cover 300 onto needle hub 210. A retaining bump 213 can also be formed in channel 212 near, but spaced from, stop 214. Retaining bump 213 can function to retain needle cover 300 in a distalmost position relative to needle hub 210 to thereby maintain the distal tip of needle 220 within needle cover 300.

In some embodiments, a proximal end of needle hub 210 may include gripping regions 216. For example, gripping regions 216 can be flattened regions on opposing sides of needle hub 210. In some embodiments, gripping regions 216 may include protrusions or other texture elements to enhance grip. The proximal end of needle hub 210 may also form a flash chamber. For example, needle hub 210 may be formed of a transparent or translucent material that allows blood within the flash chamber to be visible to the clinician. Needle hub 210 may also include a vent plug (not visible) to vent air from the flash chamber as blood flows towards and into the flash chamber.

FIGS. 4A-4D provide various views of needle cover 300 in isolation. Needle cover 300 includes a channel 301 that extends lengthwise along the top of needle cover 300 from a proximal end 300p to a distal end 300d. In this context, “top” refers to the side of needle cover 300 that would typically be facing upward during use of IV catheter device 10. Channel 301 allows push tab 111 to extend out through needle cover 300 and allows catheter adapter 110 to slide relative to needle cover 300. Grips 302 may be formed at proximal end 300p to facilitate sliding needle cover 300 relative to needle hub 210. For example, grips 302 can extend from opposing sides of needle cover 300 and may be rounded to improve comfort.

Needle cover 300 may also include guides 304 that extend inwardly within needle cover 300 and that may be positioned at or near proximal end 300p. Guides 304 can be shaped and sized to fit within channels 212 on needle hub 210. To couple needle cover to needle hub 210, guides 304 can be aligned with channels 212 and then forced over stops 214. As stated above, the ramped distal side of stops 214 can facilitate sliding guides 304 over stops 214 and into channels 212. In some embodiments, guides 304 can include a ramped proximal side to further facilitate this sliding.

In some embodiments, needle cover 300 may be formed of a plastic or other relatively hard material that cannot be punctured by needle 220, including when needle cover 300 is functioning as a needle shield. Also, the diameter or other outer dimension of needle shield 300 can be sufficient to prevent needle 220 from extending through channel 301.

FIG. 5A is a cross-sectional view of IV catheter device 10 in the pre-use configuration. As shown, tapered tip 211 of needle hub 210 is inserted into catheter adapter 110. Although not visible, protrusion 217 is inserted into notch 112 to thereby prevent catheter adapter 110 from rotating relative to needle hub 210. Also, needle cover 300 is in its distalmost position relative to needle hub 210 and encloses the distal tip of needle 220. In this distalmost position, guides 304 are positioned between stops 214 and retaining bumps 213. Guides 304 will contact retaining bumps 213 to thereby prevent needle cover 300 from sliding proximally relative to needle hub 210 until a clinician applies a sufficient, intentional force.

FIG. 5B is a cross-sectional view showing how guides 304 engage with stops 214 and retaining bumps 213 within channels 212. As shown, retaining bumps 213 extend sufficiently into channels 212 to prevent guides 304 from bypassing retaining bumps 213 without being deflected outwardly. In some embodiments, to facilitate this outward deflection when an intentional force is applied to slide needle cover 300 proximally relative to needle hub 210, the proximal side of guides 304 and the distal side of retaining bumps 213 may be ramped. Also, in some embodiments, the distal side of guides 304 and the proximal side of retaining bumps 213 may also be ramped to facilitate deflection of guides 304 when needle cover 300 is slid distally relative to needle hub 210 to return needle cover 300 to its distalmost position. In contrast, the proximal side of stops 214 may not be ramped to prevent guides 304 from escaping distally from channels 212. Likewise, the distal side of stops 215 may also not be ramped to prevent guides 304 from escaping proximally from channels 212

FIGS. 6A-6D illustrate how IV catheter device 10 may be used and particularly how needle cover 300 can also be used as a needle shield. FIG. 6A, like FIG. 1, shows IV catheter device 10 in the pre-use configuration. In this configuration, guides 304 can be positioned between stops 214 and retaining bumps 213 to prevent the distal tip of needle 220 from being unintentionally exposed.

In FIG. 6B, needle cover 300 has been slid proximally relative to needle hub 210 to thereby expose needle 220 and catheter 120. For example, a clinician could use grips 302 and/or gripping regions 216 to apply sufficient force to cause guides 304 to deflect outwardly and overtop retaining bumps 213 and to then slide proximally within channels 212 to sufficiently expose needle 220 and catheter 120. In some embodiments, as guides 304 bypass retaining bumps 213, tactile feedback and/or audible feedback (e.g., a snap) may occur. Notably, at this step, needle hub 210 and catheter adapter 110 may remain interlocked so that needle 220 extends distally from catheter 120. Accordingly, a clinician can insert needle 220 and catheter 120 into a patient's vasculature with IV catheter device 10 in this configuration.

In FIG. 6C, it can be assumed that the clinician has inserted needle 220 and catheter 120 into the patient's vasculature and has then used push tab 111 to advance catheter adapter 110 and therefore catheter 120 distally relative to needle 220 to thereby position catheter 120 appropriately within the patient's vasculature. Notably, because of channel 301, catheter adapter 100 can slide within and independent of needle cover 300. At this step, needle cover 300 can remain in its proximal position relative to needle hub 210.

In FIG. 6D, needle hub 210 has been slid proximally relative to needle cover 300 to thereby return needle cover 300 to its distalmost position. For example, the clinician could use gripping regions 216 and grips 302 to pull needle hub 210 proximally until guides 304 bypass retaining bumps 213 to thereby lock needle cover 300 in its distalmost position. The clinician may do so prior to pulling needle 220 out from catheter adapter 110 so that the distal tip of needle 220 does not become exposed. In some embodiments, as guides 304 bypass retaining bumps 213, tactile feedback and/or audible feedback may occur to thereby confirm to the clinician when needle cover 300 is secured in its distalmost position.

At this point, needle cover 300 functions as a needle shield thereby allowing needle assembly 200 to be safely disposed. Also, because needle 220 is contained within needle cover 300 when it is pulled out from catheter adapter 110, the risk of blood splatter is greatly reduced. In particular, any blood that may escape needle 220 would be contained within needle cover 300.

FIGS. 7A-7D provide an example where catheter adapter 110 includes wings and needle cover 300 is configured to accommodate the wings. In this example, IV catheter device 10 may otherwise be similar to the embodiments described above. FIG. 7A illustrates catheter adapter 110 in isolation, and FIG. 7B illustrates needle cover 300 in isolation. FIGS. 7C and 7D are perspective and distal views of IV catheter device 10 respectively.

In such embodiments, opposing wings 113 may extend outwardly from the body of catheter adapter 110. Wings 113 may be positioned on or towards the bottom of catheter adapter 110 (e.g., opposite push tab 111) so that they may be used as a securement platform. Corresponding wing channels 305 may be formed along needle cover 300 to accommodate wings 113 as needle cover 300 is slid relative to catheter assembly 100.

FIGS. 8A-8D provide another example of how IV catheter device 10 can be configured in accordance with one or more embodiments of the present disclosure. In the depicted embodiment, needle cover 300 includes a channel 301 as described above except that a deviation 301a is formed towards distal end 300d. Deviation 301a in channel 301 may function to prevent needle cover 300 from being withdrawn from catheter adapter 100 without rotating needle cover 300 relative to catheter adapter 110. In particular, absent such rotation, push tab 111 will contact a distal wall of deviation 301a to block further distal movement of catheter assembly 100 relative to needle cover 300. As a result, the clinician will need to perform such rotation to separate needle shield 300 from catheter adapter 110, and therefore to withdraw needle 220 from catheter adapter 110 (e.g., as shown in FIGS. 6C and 6D). This requirement for rotation can remind the clinician to slide needle shield 300 into its distalmost position relative to needle hub 210 before withdrawing needle 220 from catheter adapter 110.

In the embodiments depicted in FIG. 8A, grips 302 are positioned in a middle section of needle cover 300 and are in the form of raised ridges or another raised pattern. In some embodiments, this raised pattern could be in the shape of an arrow pointing proximally to thereby inform the clinician that needle shield 300 is to be withdrawn proximally relative to catheter assembly 100 to expose needle 220. In some embodiments, an arrow 218 or other directional indicia may be formed on the proximal end of needle hub 210 and may function to provide guidance to the clinician regarding the rotation of needle hub 210 relative to needle shield 300 as described below.

As best shown in FIGS. 8B-8D, each channel 212 includes a locking slot 212a that extends from the side of channel 212 at retaining bump 213. In other words, locking slots 212a are configured to receive guides 304 when guides 304 have passed distally beyond retaining bumps 213 and needle hub 210 is rotated relative to needle shield 300. Once guides 304 are positioned in locking slots 212a, needle shield 300 will be prevented from separating from needle hub 210. In particular, locking slots 212a, which form proximal and distal walls around guides 304, minimize the likelihood that needle shield 300 will separate from needle hub 210 when compressive and tensile loads are applied. As best seen in FIG. 8C, locking slots 212a may be recessed from channel 212 to form a step that prevents guides 304 from being rotated back into channel 212. FIGS. 8B-8D also show that much of the structure of needle hub 210 surrounding tapered tip 211 can be eliminated to reduce the amount of material that is required to manufacture needle hub 210. Furthermore, the proximal ends of channels 212 may include a retaining bump 215a that functions in a similar manner as retaining bump 213.

IV catheter device 10 can be packaged in the configuration represented in FIG. 8A in which guides 304 are positioned distal to retaining bumps 213. Notably, due to the interface between protrusion 217 and notch 112, needle hub 210 cannot rotate independent of catheter adapter 110. Also, because push tab 111 is rotationally confined within channel 301, needle hub 210 cannot rotate relative to needle shield 300 thereby preventing guides 304 from entering locking slots 212a while IV catheter device 10 is in this initial packaged state.

To use IV catheter device 10, the clinician can use grips 302 to pull needle shield 300 relative to needle hub 210 to thereby cause guides 304 to pass proximally overtop retaining bumps 213 and slide within channels 212 until passing over retaining bumps 215a (e.g., similar to what is shown in FIG. 6B). In some embodiments, an audible snap may be created as guides 304 pass over retaining bumps 215a. This proximal movement of needle shield 300 will expose the distal end of needle 220. Retaining bumps 215a can be configured so that the force required to pass guides 304 distally over retaining bumps 215a is greater than the force required to puncture the skin. As a result, the clinician can grasp needle shield 300 while inserting needle 220 without the risk that needle shield 300 will slide distally relative to needle hub 210.

Once needle 220 is inserted, the clinician can push catheter adapter 110 distally relative to needle hub 210 and needle shield 300 to separate needle hub 210 from catheter adapter 110 (e.g., similar to what is shown in FIG. 6C). The clinician may also pull needle hub 210 proximally relative to needle shield 300 until guides 304 have passed distally beyond retaining bumps 213 thereby causing needle shield 300 to cover the distal tip of needle 220. At this point, the clinician can rotate needle shield 300 relative to needle hub 210 to position guides 304 in locking slots 212a. The clinician may also rotate needle shield 300 relative to catheter adapter 110 to guide push tab 111 through deviation 301a and out from channel 301 (e.g., similar to what is shown in FIG. 6D). The clinician may then safely dispose of needle assembly 200 and needle shield 300.

FIG. 9 illustrates an example where tab portion 111 is elongated to create a pushing surface. In such embodiments, the top of tab portion 111 may be inclined distally to improve grip.

FIG. 10 provides an example where gripping regions 216 extend distally overtop channels 212. In such embodiments, the length of needle hub 210 can be reduced given that gripping regions 216 are not formed proximal to channels 212. In such embodiments, needle hub 210 could be manufactured as two separate components, a distal component and a proximal component that includes gripping regions 216, that could be assembled using a snap interface, plastic welding, threading, an interference fit, etc.

FIGS. 11A-11D provide another example of how IV catheter device 10 can be configured in accordance with one or more embodiments of the present disclosure. In the depicted embodiment, needle cover 300 includes a channel 301 with a deviation 301a as described above except that deviation 301a is formed towards proximal end 300p. In the depicted embodiment, each channel 212 of needle hub 210 includes an entry 212b that extends alongside the distal end of channel 212. A first one-way bump 212c may divide entry 212b and a second one-way bump 212d may divide entry 212b from channel 212. Needle hub 210 may also include one or more cutouts 219 for receiving support tabs 306 (see FIG. 12D) in needle shield 300.

Entry 212b facilitates assembly of IV catheter device 10. In particular, to couple needle shield 300 to needle hub 210, support tab(s) 306 and guides 304 can be aligned with cutout(s) 219 and entries 212b respectively and then guides 304 can be inserted into entries 212b until passing over one-way bumps 212c as shown in FIG. 13. One-way bumps 212c can have a flat proximal face that prevents guides 304 from passing distally over them. Needle shield 300 can remain in this position relative to needle hub 210 while packaged.

To use IV catheter device 10, needle shield 300 can be rotated relative to needle hub 210 to pass guides 304 over one-way bumps 212d and into channel 212. The proximal end of needle hub 210 can be configured to not block support tab(s) 306 as needle shield 300 is rotated (e.g., by eliminating material between channel 212 and the opposite entry 212b). Needle shield 300 may then be slid proximally relative to needle hub 210 to pass guides 304 over retaining bumps 215a to expose needle 220. Once needle 220 is inserted and catheter adapter 110 is advanced, which may include guiding push tab 111 through deviation 301a, needle shield 300 can be slid distally relative to needle hub 210 until guides 304 pass over retaining bumps 213 as shown in FIG. 11D. In this position, support tab(s) 306 may abut a proximal wall of needle hub 210 to better reinforce the coupling of needle hub 210 and needle shield 300. At this point, needle hub 210 and needle shield 300 may be safely disposed.

FIGS. 12A-12D illustrate an embodiment of needle shield 300 that may be used in conjunction with the embodiment of IV catheter device 10 shown in FIGS. 11A-11D. Needle shield 300 includes channel 301 having deviation 301a at proximal end 300p as well as a distal opening 301b that selectively opens when push tab 111 is intentionally forced through it. Notches 307 may be formed on opposing sides of distal opening 301b to facilitate this selective opening by allowing distal opening 301b to flex open. As shown in FIG. 12C, in some embodiments, the proximal end of channel 301 may be widened to facilitate insertion of push tab 111 during assembly of IV catheter device 10.

FIGS. 14A-14F provide another example of a needle shield 300 and needle hub 210 that may be used in embodiments of the present disclosure. The depicted needle shield 300 is similar to the embodiment represented in FIGS. 12A-12D with the addition of notches 308 at proximal end 300p. One-way bump 212c of needle hub 210 also includes a ramped distal surface 212c1 that interacts with guides 304 to gradually widen proximal end 300p to allow guides 304 to pass overtop one-way bump 212c. Notches 308 can facilitate this widening of proximal end 300p at guides 304. FIG. 14E represents IV catheter device 10 in its packaged configuration, while FIG. 14F represents IV catheter device 10 in its post-insertion locked configuration.

As can be seen, an IV catheter device configured in accordance with embodiments of the present disclosure provides a simplified design without sacrificing safety. By configuring the needle cover to function also as a needle shield, complex and expensive needle shield components are not needed. Also, the open design of the needle cover facilitates sterilization of the IV catheter device.

Embodiments of the present disclosure encompass a number of variations from the depicted embodiments. For example, in some embodiments, one or more channels could be formed in needle cover 300 and corresponding guide(s) could be formed on needle hub 210. As another example, in some embodiments, the cross-sectional shape of channels 212 could be another suitable shape such as square, triangular, circular, etc.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. An IV catheter device comprising:
a catheter assembly having a catheter adapter and a catheter that extends distally from the catheter adapter;
a needle assembly having a needle hub and a needle that extends distally from the needle hub, the needle hub being coupled to the catheter adapter such that the needle extends through the catheter; and
a needle cover that is coupled to the needle hub, the needle cover being configured to function as a needle shield after the needle is withdrawn from the catheter adapter.

2. The IV catheter device of claim 1, wherein the needle cover is coupled to the needle hub via one or more channels and corresponding one or more guides.

3. The IV catheter device of claim 2, wherein the one or more channels extend along the needle hub and the corresponding one or more guides are formed on the needle cover.

4. The IV catheter device of claim, 3, wherein each of the one or more channels includes a distal stop and a retaining bump spaced from the distal stop.

5. The IV catheter device of claim 4, wherein each of the one or more channels includes a proximal stop.

6. The IV catheter device of claim 2, wherein each of the one or more channels includes a locking slot into which the one or more guides are rotated.

7. The IV catheter device of claim 2, wherein the needle hub includes one or more entries corresponding to the one or more channels by which the one or more guides enter the one or more channels.

8. The IV catheter device of claim 1, wherein the needle cover includes a channel and the catheter adapter includes a push tab that extends through and slides within the channel.

9. The IV catheter device of claim 8, wherein the channel includes a deviation.

10. The IV catheter device of claim 1, wherein the needle hub includes one or more cutouts and the needle shield includes one or more support tabs that insert through the one or more cutouts to assemble the needle hub and needle shield.

11. The IV catheter device of claim 1, wherein the catheter adapter includes wings and the needle cover includes wing channels within which the wings slide.

12. An IV catheter device comprising:
a catheter assembly having a catheter adapter and a catheter that extends distally from the catheter adapter;
a needle assembly having a needle hub and a needle that extends distally from the needle hub, the needle hub being coupled to the catheter adapter such that the needle extends through the catheter; and
a needle cover that is coupled to the needle hub and configured to slide along the needle hub from a distalmost position to a proximal position, wherein the needle cover encloses the needle and the catheter when the needle cover is in the distalmost position and the catheter adapter is coupled to the needle hub, and wherein the needle cover encloses the needle when the needle cover is in the distalmost position and the catheter assembly has been slid distally out from the needle cover.

13. The IV catheter device of claim 12, wherein the needle cover includes a channel and the catheter adapter includes a push tab that extends through the channel, the push tab enabling a clinician to slide the catheter adapter distally out from the needle cover.

14. The IV catheter device of claim 12, wherein the needle hub includes one or more channels and the needle cover includes corresponding one or more guides that slide within the one or more channels.

15. The IV catheter device of claim 14, wherein each of the one or more channels includes a retaining bump that interfaces with the corresponding guide to retain the needle cover in the distalmost position.

16. The IV catheter device of claim 15, wherein each of the one or more channels includes a distal stop that is spaced from the retaining bump.

17. The IV catheter device of claim 12, wherein the catheter adapter includes a notch and the needle hub includes a protrusion that inserts into the notch to thereby limit rotation of the catheter adapter relative to the needle hub.

18. An IV catheter device comprising:
a catheter assembly having a catheter adapter and a catheter that extends distally from the catheter adapter, the catheter adapter including a push tab;
a needle assembly having a needle hub and a needle that extends distally from the needle hub, the needle hub being coupled to the catheter adapter such that the needle extends through the catheter, the needle hub having one or more channels that extend lengthwise along the needle hub; and
a needle cover having one or more guides that are positioning within the one or more channels to thereby couple the needle cover to the needle hub, the one or more guides being configured to slide within the respective channel, the needle cover including a channel within which the push tab of the catheter adapter slides.

19. The IV catheter device of claim 18, wherein each of the one or more channels includes a distal stop and a retaining bump, the respective guide being selectively retained between the distal stop and the retaining bump.

20. The IV catheter device of claim 19, wherein each of the one or more channels includes a locking slot into which the respective guide is rotated.
